(19) United States
(12) Patent Application Publication
(10) Pub. No.: US 2020/0215231 A1
(43) Pub. Date: Jul. 9, 2020

(54) NOVEL XENOGRAFT

(71) Applicant: BONE LIGAMENT TENDON PTY LTD, Bowral (AU)

(72) Inventor: Nicholas James Hartnell, Bowral (AU)

(73) Assignee: BONE LIGAMENT TENDON PTY LTD, Bowral (AU)

(21) Appl. No.: 16/631,238

(22) PCT Filed: Jul. 17, 2018
(86) PCT No.: PCT/AU2018/000116

§ 371 Date: Jan. 15, 2020

(30) Foreign Application Priority Data
Jul. 17, 2017 (AU) 2017902800

(51) Int. Cl. A61L 27/36; A61F 2/08; A61L 27/50

(52) U.S. Cl. CPC A61L 27/3604; A61F 2/08; A61L 2430/10; A61L 27/3695; A61L 27/50; A61L 27/3662

(57) ABSTRACT

A xenograft for treating a tendon or ligament condition in a subject, wherein said xenograft comprises at least a portion of a macropod tendon.

### FIELD OF THE INVENTION

The present invention relates to xenografts for repair of ligaments and tendons.

### BACKGROUND

Trauma to a ligament or tendon may result in loss of either stability and/or movement of the associated joint. In many cases the tendon or ligament will “scar” appropriately without surgical reconstruction and the function will adequately return. In noticeable exceptions, that does not occur. This is most evident in tendons that are intra-articular (inside the joint), such as the anterior cruciate ligament (ACL) in the knee. It is also evident in the flexor tendons to the hand and foot and the ligaments in the ankle. Chronic or delayed treatment of the Achilles tendon or the quadriceps tendon in humans also gives rise to inadequate healing and thus failure of movement or function. To repair or reconstruct these tendons or ligaments, it is necessary to implant a substitute.

Existing options for substitution include autografts, allografts and synthetic substitutes. An autograft is tendon, most commonly the hamstrings, or other strip of soft tissue or fascia taken from the patient. An allograft is cadaveric tendon or ligament. Problems arise for all of these options. In the case of autografts, there is morbidity in taking healthy tissue from an uninjured area. Allografts are mostly sourced from elderly cadavers and therefore suffer from compromised strength, in addition to there being infection risks and limited supply. Synthetic substitutes often fail with further issues and complication for the patient, such as synovitis from debris.

A further option is a xenograft, which is a graft transferred from one animal species to another. Xenografts are potentially able to solve a number of the aforementioned issues and may therefore be of significant surgical benefit for surgeons and patients. There is therefore a need for new xenografts having suitable biomechanical properties for repairing injured tendons, ligaments or both.

### SUMMARY OF THE INVENTION

According to a first aspect of the present invention there is provided a xenograft for treating a treating a tendon or ligament condition in a subject, wherein said xenograft comprises at least a portion of a macropod tendon.

The following options may be used in combination with the above aspect, either individually or in any suitable combination.

The macropod tendon may be an Achilles tendon or it may be a tail tendon. The macropod may be selected from the group consisting of red kangaroo (Macropus rufus), eastern grey kangaroo (Macropus giganteus), western grey kangaroo (Macropus fuliginosus), black wallaroo (Macropus bernardus), antilopine wallaroo (Macropus antilopinus), common wallaroo (Macropus robustus), agile wallaby (Macropus agilis), black-striped wallaby (Macropus dorsalis), red-necked wallaby (Macropus rufogriseus), swamp wallaby (Wallabia bicolor) and whiptail wallaby (Macropus parryi). The macropod may be selected from the group consisting of red kangaroo (Macropus rufus), eastern grey kangaroo (Macropus giganteus) and western grey kangaroo (Macropus fuliginosus). The may be an eastern grey kangaroo (Macropus giganteus).

The subject may be a mammal. The mammal may be a human. The xenograft may be non-immunogenic. It may have been subjected to a denaturing process.

The xenograft may comprise a portion of macropod tendon having an ultimate tensile strength of at least about 10 MPa, at least about 20 MPa or at least about 30 MPa. The xenograft may comprise a portion of macropod tendon having an elastic modulus of at least about 50 MPa, at least about 100 MPa or at least about 200 MPa.

According to a second aspect of the present invention there is provided a method for treating a tendon or ligament condition in a mammal comprising implanting the xenograft of the first aspect of the invention at the site of said tendon or ligament.

The following options may be used in combination with the above aspect, either individually or in any suitable combination.

The tendon or ligament condition may be a tendon injury or ligament injury. The tendon or ligament may be selected from the group consisting of anterior cruciate ligament (ACL), posterior cruciate ligament (PCL), lateral collateral ligament (LCL), medial collateral ligament (MCL), patellar ligament, palmar radiocarpal ligament, dorsal radiocarpal ligament, ulnar collateral ligament, radial collateral ligament, Achilles tendon, flexor tendon of the hand, flexor tendon of the foot, extensor tendon of the hand, extensor tendon of the foot, rotator cuff tendons of the shoulder, hip abductor tendons, deltoid ligament complex of the ankle, lateral ligament complex of the ankle, calcaneofibular ligament, patella tendon, quadriceps tendon, medial patellofemoral ligament of the knee and lateral patellofemoral ligament of the knee.

The strength of the at least a portion of the macropod tendon may be equal to or greater than the strength of a healthy native tendon or ligament. The elastic modulus of the at least a portion of the macropod tendon may be equal to or greater than the elastic modulus of a healthy native tendon or ligament.

According to a third aspect of the present invention there is provided use of the xenograft of the first aspect of the invention for treating a tendon or ligament in a mammal.

### DESCRIPTION OF FIGURES

Embodiments of the present invention will now be described, by way of example only, with reference to the accompanying figures wherein:

FIG. 1 is a schematic diagram showing the dissection of kangaroo Achilles tendon specimens;

FIG. 2 is a schematic diagram showing the dissection of human ACL specimens;

FIG. 3 is a schematic diagram showing the point of failure (*) and the three biomechanics outcome measures (ultimate tensile strength, elastic modulus and strain at failure) calculated from tensile stress-strain data;

FIG. 4 shows (a) ultimate tensile strength (mean±STD); (b) elastic modulus (mean±STD); and (c) strain at failure (mean±STD) of the proximal and distal portions of kangaroo tendon and the AM and PL bundles of human ACL (* indicates p<0.05 for comparisons between locations within each tissue type);

FIG. 5 shows (a) ultimate tensile strength (mean±STD); (b) elastic modulus (mean±STD) of kangaroo tendon and human (# indicates p<0.05);

FIG. 6 shows representative images of Hematoxylin-and-Eosin stained (A-H) and Toluidine Blue stained (I-L) sections from proximal (A, E, I) and distal (B, F, J) kangaroo Achilles tendon and anterior (C, G, K) and posterior (D, H, L) human ACL (images E-H are higher magnification of regions of images A-D; arrows indicate inter-fibrillar space and cells that were more prominent in kangaroo Achilles tendon);

FIG. 7 shows the comparative cell numbers (per high-power field) in anterior and posterior human ACL versus proximal and distal kangaroo Achilles tendon (n=6-8 specimens/group);

FIG. 8 shows representative polarised light microscopy images of Picrosirius red stained sections from proximal (A, E, I) and distal (B, F, J) kangaroo Achilles, and anterior (C, G, K) and posterior (D, H, L) human ACL (the variability in collagen fibre alignment from maximum (dark) to minimum (light) between samples of a given tissue are shown in each row);

FIG. 9 shows representative images immunostaining for collagen type I (A-E) and III (F-J) in sections from proximal and distal kangaroo Achilles and human ACL; and

FIG. 10 shows representative images of elastin micro-fibres (black, as indicated by the arrows) in sections from different specimens of proximal (A, B) and distal (C, D) kangaroo Achilles and anterior (E-G) and posterior (H-J) human ACL (inter-fibrillar elastin running at an angle to the collagen seen exclusively in ACLs is shown in K, and can be compared with thicker fibres in elastic tissues such as aorta in L).

### DETAILED DESCRIPTION

Described herein are xenografts and methods of treating tendon and ligament conditions in subjects using those xenografts.

In the context of this specification, the term “comprising” means “including principally, but not necessarily solely”. Furthermore, variations of the word “comprising”, such as “comprise” and “comprises”, have correspondingly varied meanings.

In the context of this specification, the term “native tendon or ligament” means a tendon or ligament in a recipient which the xenograft is intended to replace or repair.

In the context of this specification, the term “xenograft” means a tissue graft from a donor of a different species from the intended recipient. The term “xenograft” encompasses tissue grafts which have been surgically removed from the donor and which are yet to be implanted into the recipient.

The xenografts described herein comprise at least a portion of an Achilles tendon from a macropod or at least a portion of a tail tendon from a macropod. It has been surprisingly found that macropod tendons provide a readily available xenograft having excellent biomechanical properties, suitable for treating a range of tendon and ligament conditions in mammals. For example, a comparative analysis of kangaroo Achilles tendon specimens against human ACL specimens show that the strength of the kangaroo Achilles tendon is approximately triple that of the human ACL. Macropod tendons are also relatively simple to harvest (e.g., compared with ligament tissue). There is therefore a significant benefit in using kangaroo Achilles tendon as a xenograft for ACL and other tendon and ligament conditions in humans and other mammals.

The xenograft described herein may be used to treat any suitable tendon or ligament condition. The xenograft described herein may be used to treat an acute or chronic tendon or ligament condition. The xenograft may be used to treat an injured (e.g., ruptured) tendon or ligament, it may be used to treat a tendon of a diseased joint (e.g., an inflammatory condition such as rheumatoid arthritis or osteoarthritis), it may be used as to replace a tendon or ligament that is absent (e.g., a congenitally absent tendon or ligament), or it may be used to fully or partially replace a malformed tendon or ligament.

The Achilles tendon or tail tendon of the macropod, or the portion thereof, is preferably healthy and undamaged. It preferably shows no signs of previous trauma. The tendon or portion thereof may have one or more biomechanical properties that are substantially equal to or better than one or more biomechanical properties of a healthy native tendon or ligament. For example, the ultimate tensile strength of the tendon or portion thereof may be equal to or greater than the ultimate tensile strength of a healthy native ligament or tendon, the elastic modulus of the tendon or portion thereof may be equal to or greater than the elastic modulus of a healthy native tendon or ligament, or both. In embodiments, the ultimate tensile strength of the tendon or portion thereof, as measured by the method described in the Examples herein, may be at least about 10 MPa, at least about 20 MPa or at least about 30 MPa. In embodiments, the elastic modulus of the tendon or portion thereof, as measured by the method described in the Examples herein, may be at least about 50 MPa, at least about 100 MPa or at least about 200 MPa.

Where the xenograft comprises only a portion of the Achilles tendon or tail tendon of the macropod, the portion may be a proximal portion or a distal portion of the tendon. The term “proximal” and “distal” as used herein refer to the distance from the centre of the animal (e.g., proximal tissue from the Achilles tendon is further away from the ankle joint than distal tissue). In embodiments, the xenograft comprises a proximal portion of a macropod Achilles or tail tendon.

The Achilles tendon or tail tendon of the macropod may be from any suitable macropod. The suitability of a macropod may be determined by the desired biomechanical properties of the tendon. For example, the macropod may be chosen to maximise the strength of the tendon, to maximise the elasticity of the tendon, to maximise the strain at failure of the tendon or to provide a balance between these biomechanical properties. The suitability of a macropod may be determined by the availability or the abundance of the species. In embodiments, the macropod may be a kangaroo, wallaroo or wallaby. For example, the macropod may be a red kangaroo (Macropus rufus), eastern grey kangaroo (Macropus giganteus), western grey kangaroo (Macropus fuliginosus), black wallaroo (Macropus bernardus), antilopine wallaroo (Macropus antilopinus), common wallaroo (Macropus robustus), agile wallaby (Macropus agilis), black-striped wallaby (Macropus dorsalis), red-necked wallaby (Macropus rufogriseus), swamp wallaby (Wallabia bicolor) or whiptail wallaby (Macropus parryi). In some embodiments, the macropod is a red kangaroo, eastern grey kangaroo or western grey kangaroo.

The macropod may be a male macropod or a female macropod. A xenograft from either a male or female macropod may be used to treat a tendon or ligament condition in a female patient. A xenograft from either a male or female macropod may be used to treat a tendon or ligament condition in a male patient. The macropod may be any suitable age. The macropod may be a juvenile macropod or an adult macropod. The macropod may be a young adult macropod.

The xenograft may be used for treating a tendon or ligament condition in any suitable subject, other than the species from which the xenograft is sourced. Thus, there is provided a xenograft for treating a tendon or ligament condition in a subject. In embodiments, the subject may be a mammal. In embodiments, the mammal may be a human. In embodiments, there is provided a xenograft for treating a tendon or ligament condition in a human.

The xenograft may be used to repair any suitable ligament or tendon in a mammal. For example, the ligament or tendon may be an anterior cruciate ligament (ACL), posterior cruciate ligament (PCL), lateral collateral ligament (LCL), medial collateral ligament (MCL), patellar ligament, palmar radiocarpal ligament, dorsal radiocarpal ligament, ulnar collateral ligament, radial collateral ligament, Achilles tendon, flexor tendon of the hand, flexor tendon of the foot, extensor tendon of the hand (including the thumb), extensor tendon of the foot (including the great toe), rotator cuff tendons of the shoulder (e.g., supraspinatus tendon, subscapularis tendon, infraspinatus tendon or teres major tendon), hip abductor tendons (e.g., gluteus maximus tendon, gluteus medius tendon or gluteus minimus tendon), deltoid ligament complex of the ankle, lateral ligament complex of the ankle (e.g., anterior talofibular ligament and posterior talofibular ligament), calcaneofibular ligament, patella tendon, quadriceps tendon, medial patellofemoral ligament of the knee or lateral patellofemoral ligament of the knee. In some embodiments, the ligament is an anterior cruciate ligament (ACL).

In preparing the xenograft for use, the tendon is surgically dissected and removed from the paratenon (tendon sheath). The tendon is composed mostly of an extra-cellular collagen matrix, which is non-immunogenic. The tendon may therefore require no denaturing (decellularisation) prior to implantation into a mammal. In some embodiments, the tendon may be subjected to a decellularisation process prior to implantation. In removing the cells from the tendon, the immunogenic antibodies within the cells are also removed, thus rendering the tendon non-immunogenic. Methods of decellularisation of tendon tissue are known in the art. Such methods include physical, chemical and enzymatic treatments, wherein the cells within the tendon are lysed, leaving an undamaged extracellular matrix having the same physical and biochemical properties as the natural tissue.

In use, the xenograft may be implanted in the subject at the site of a damaged ligament or tendon in order to repair the damage. The xenograft may be implanted by any method for xenograft implantation known in the art. A method is thus provided for treating a tendon or ligament condition in a subject comprising implanting the xenograft described herein at the site of a damaged ligament or tendon. In embodiments, the subject may be a mammal. In embodiments, the mammal may be a human. In embodiments, there is provided a method for treating a tendon or ligament condition in a human comprising implanting the xenograft described herein at the site of a damaged ligament or tendon.

### EXAMPLES

Kangaroo Achilles tendon was evaluated to determine its suitability as a tendon/ligament xenograft material in humans. The analyses consisted of biomechanical and histological comparison with human anterior cruciate ligaments (ACLs).

Twelve male eastern grey kangaroo Achilles tendons were analysed (<12 hours after death). These specimens were dissected into proximal and distal portions and trimmed to lengths of 30-35 mm. Each of the specimens was then further longitudinally divided into portions for different analyses; biomechanics (2 specimens), histology, and electron microscopy (FIG. 1).

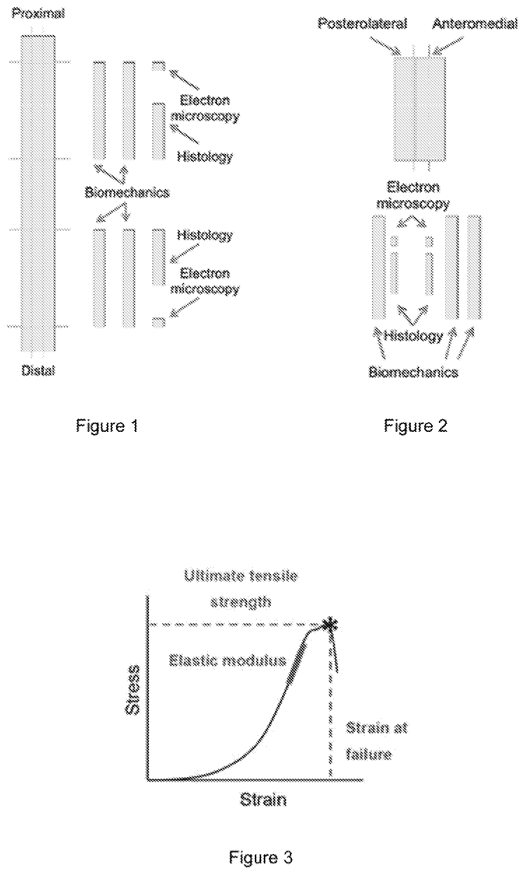

Ten knees from five male human cadaveric donors (age 66-74 years) were procured by the Murray Maxwell Biomechanics Laboratory. Male specimens were chosen to match the sex of the human specimens with the sex of the kangaroo specimens. Knees were defrosted and dissected to expose the ACL, which was then dissected from its bony attachments. It was necessary to remove the bony attachments and dissect the ACL into smaller test specimens to match the testing conditions between the kangaroo tendons and human ACLs. The ACLs were divided into anteromedial (AM) and posterolateral (PL) bundles and then further divided longitudinally into specimens for biomechanics, histology and electron microscopy (FIG. 2). When the ACLs were harvested, the PL bundles were shorter and more difficult to isolate, therefore it was only possible to dissect one biomechanics specimen from the PL bundle of each ACL instead of two (FIG. 2).

All specimens for biomechanics were wrapped in saline soaked gauze as soon as possible after dissection and then stored at −20° C. prior to testing.

### Example 1. Biomechanical Testing and Analysis

Method

The specimens allocated to biomechanical testing were thawed to room temperature and trimmed into specimens with consistent cross-sectional area and length. The cross-sectional area of the specimens was measured using a micrometer device. The proximal and distal ends of the specimens were wrapped in dry cardboard to prevent slippage of the specimen in the grips during testing. The cardboard ends were then clamped in pneumatic, sandpaper-lined grips (10 mm on each side, leaving ˜10 mm exposed between the grips for testing) and tested under tension (stretched) at 5 mm/s (˜0.5/s strain rate) until failure occurred (indicated by a sharp drop in force). The tension (force) in the specimen during testing was measured at 100 Hz using a 250N load cell. The displacement of the grips was measured by the Instron testing machine at 100 Hz and video was captured during testing at 2 Hz to inspect for slippage of the specimen at the grips. The length of the specimen exposed for testing was measured as the distance between the grips when the force exceeded 1N. This ensured an objective and consistent determination of ‘initial length’ (or ‘gauge length’) across all specimens. The force data were normalised by the specimen cross sectional area (i.e. converted to ‘stress’) and the displacement data were normalised by the initial length (i.e. converted to ‘strain’). Stress versus strain curves were developed to measure three outcome variables: elastic modulus, ultimate tensile strength and strain at failure. The elastic modulus (or ‘stiffness’) is the gradient of the stress-strain curve and provides an indication of the level of ‘resistance to stretch’. Biological materials have non-linear stress-strain curves, so the location and method of measuring the elastic modulus can vary. The elastic modulus was calculated for the entire length of each stress-strain curve and the maximum recorded. The maximum stress is the point of failure of the tendon and at this point the stress and strain values were recorded, which are the ultimate tensile strength and the strain at failure respectively. The ultimate tensile strength gives an indication of how ‘strong’ the material is regardless of size and the strain at failure gives an indication of how much the specimen stretches before it fails.

All data were analysed using mixed model linear regression (using Stata v14.0) accounting for clustering on specimen ID and location (i.e. proximal/distal for tendons or AM/PL bundle for ACLs) to account for non-independence of these variables. The first part of the analysis was to determine whether the location of the test specimen affected any of the three biomechanical outcome measures, so separate analyses were performed for each tissue type (kangaroo tendon and human ACL) for each of the three biomechanics outcomes. Since there was an effect of location on strain at failure (discussed below), the location variable was included in subsequent statistical modelling. The second part of the analysis was to determine whether there are differences in biomechanical properties for kangaroo tendons and human ACLs, when tested in the same way. Mixed model regression was performed for each of the three biomechanical outcomes with tissue source (kangaroo tendon and human ACL) as an independent variable and accounting for clustering on specimen ID and location, to account for non-independence.

Results & Discussion

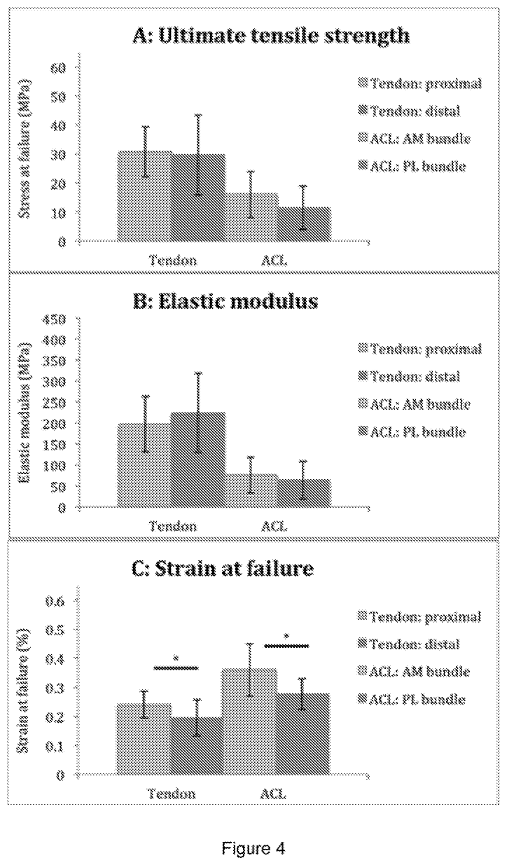

To determine if a specific region of the kangaroo Achilles tendon was better matched in biomechanical properties to a specific region of the human ACL, the proximal and distal portions of the kangaroo tendon and the AM and PL bundles of the ACL were investigated separately. The results from the statistical analysis for location of the test specimen are presented in Tables 1 and FIG. 4.

TABLE 1
Results from statistical analysis for location of test specimen

| | | |
|---|---|---|
| Co-efficient | p-Value | 95% Confidence Interval |

Strength

| | | | | |
|---|---|---|---|---|
| Tendon: proximal/ | −1.152 | 0.799 | −9.999 | 7.700 |
| distal (n = 24) | | | | |
| ACL: AM/PL bundle | −4.575 | 0.196 | −11.512 | 2.363 |
| (n = 20) | | | | |

Modulus

| | | | | |
|---|---|---|---|---|
| Tendon: proximal/ | 27.275 | 0.453 | −44.001 | 98.551 |
| distal (n = 24) | | | | |
| ACL: AM/PL bundle | −11.911 | 0.526 | −48.760 | 24.939 |
| (n = 20) | | | | |

Strain at Failure

| | | | | |
|---|---|---|---|---|
| Tendon: proximal/ | −0.045 | 0.034* | −0.087 | −0.003 |
| distal (n = 24) | | | | |
| ACL: AM/PL bundle | −0.082 | 0.008* | −0.143 | −0.021 |
| (n = 20) | | | | |

The location of the test-specimen within the tissue (i.e. proximal/distal for tendons, AM/PL bundle for ACLs) had no statistically significant effect on strength or modulus (i.e. p>0.05). When taking into account the non-independence of each specimen in the statistical modelling, there was no significant difference between the regions of the tendon or the regions of the ACL, for strength and elastic modulus. Thus, there is no specific region within the tendon (proximal or distal) that better matches the strength or ‘stiffness’ of the ACL.

In contrast to the strength modulus, the location of the specimen was found to significantly affect strain at failure. Proximal tendon had significantly higher failure strain than distal tendon (p=0.034) and AM ACL had significantly higher failure strain than PL ACL (p=0.008). The analysis also showed that human ACL had significantly higher strain at failure than the kangaroo tendon. It may therefore be preferable to use proximal tendon in comparison with the distal tendon for this specific biomechanical outcome.

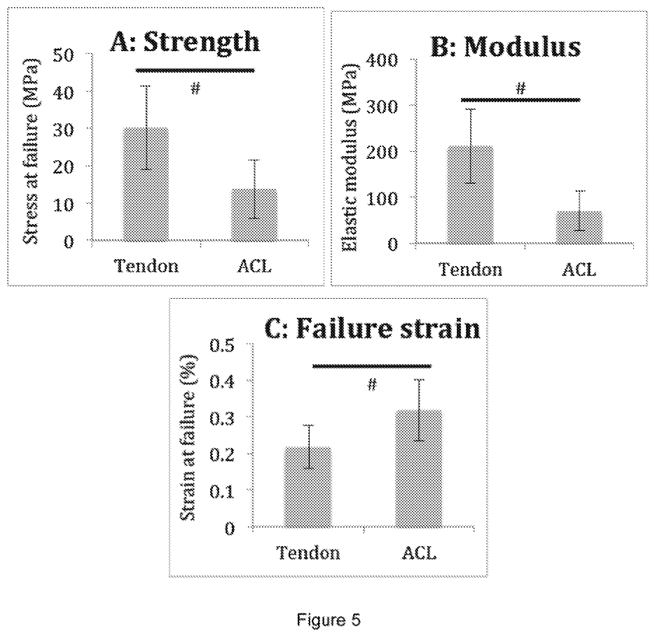

To compare biomechanical properties of the kangaroo tendon and human ACL, ultimate tensile strength, elastic modulus and strain at failure were determined for specimens of both. The results from the statistical analysis comparing kangaroo tendon and human ACL are presented in Table 2 and FIG. 5.

TABLE 2
Results from statistical analysis comparing
kangaroo tendon and human ACL

| | | |
|---|---|---|
| Co-efficient | p-Value | 95% Confidence Interval |

Strength

| | | | | |
|---|---|---|---|---|
| Tissue source/ | −16.458 | <0.001# | −22.898 | −10.018 |
| species (n = 44) | | | | |

Modulus

| | | | | |
|---|---|---|---|---|
| Tissue source/ | −140.963 | <0.001# | −185.722 | −96.204 |
| species (n = 44) | | | | |

Strain at Failure

| | | | | |
|---|---|---|---|---|
| Tissue source/ | 0.101 | 0.002# | 0.037 | 0.164 |
| species (n = 44) | | | | |

Ultimate tensile strength was significantly higher (more than double) in specimens from kangaroo Achilles tendon compared with specimens from the human ACLs tested (p<0.001). It should be noted that the human cadaveric donors supplying tissue for this study where aged 66-74 years. The ACL strength would likely be higher in younger patients, so a high ultimate tensile strength of the kangaroo Achilles tendon would be beneficial. A study of human cadaveric (intact) ACL biomechanics by Chandrashekara et al. found the stress at failure to be 26.35±10.08 MPa (mean±STD) in males aged 26-50 years (Chandrashekara N, Mansourib H, Slauterbeckc J, Hashemia J, “Sex-based differences in the tensile properties of the human anterior cruciate ligament”, Journal of Biomechanics, 39 (2006) 2943-2950). This is close to the strength of the kangaroo Achilles tendon specimens tested in this study (30.18±11.22 MPa, see FIG. 5A), confirming that the kangaroo Achilles tendon is a suitable match for the human ACL based on ultimate tensile strength.

Elastic modulus was significantly higher (approximately triple) in specimens from kangaroo Achilles tendon compared with specimens from the human ACLs tested (p<0.001). Again, it should be noted that the age of the human donors in this study may have an effect on stiffness. The aforementioned study by Chandrashekara et al. reported a higher elastic modulus for younger male donors at 128±35 MPa, which is again closer to the kangaroo tendon elastic modulus (211±80 MPa) than the older human cadaveric specimens tested in this study. This confirms that the kangaroo Achilles tendon is a suitable match for the human ACL based on elastic modulus.

Strain at failure was lower (around two thirds) in specimens from kangaroo Achilles tendon compared with specimens from the human ACLs tested (p=0.002). The observed failure strain for the human ACLs (0.32±0.08) is comparable with that observed in the Chandrashekara et al. (2006) study (0.3±0.06). The failure strain was higher for the proximal kangaroo Achilles tendon (0.24±0.05; FIG. 4C) than for the distal kangaroo Achilles tendon, making it closer in biomechanical properties to the human ACL than the distal tendon. Therefore, it may be preferable for some applications to use proximal tissue. Although the strain at failure was less for the kangaroo tendon than the human ACL, because the tendon is stronger than the human ACL it is anticipated that this would be less significant in vivo.

### Example 2. Histology and Immunohistology Study

### Method

Histo-morphology: following dissection, samples were fixed in 10% neutral buffered formalin (>24 hours), dehydrated in ethanol and infiltrated for 3 weeks in methyl-benzoate (3 changes) and paraffin under vacuum (4 days). The samples were then paraffin embedded and longitudinal sections stained with Hematoxylin-and-Eosin, Toluidine blue-and-Fast green, and Picrosirius red. All samples were evaluated qualitatively for morphology, cellularity (including cell-count/high power field), proteoglycan content (toluidine blue staining) and collagen fibre alignment (polarized light microscopy).

Electron microscopy: a sample of each kangaroo Achilles tendon specimen and human ACL specimen was fixed and stored for follow up evaluation of collagen fibre diameters. Following dissection, samples were fixed in freshly prepared paraformaldehyde followed by osmium and cacodylate infiltration. The samples were then embedded in Spurs resin and stored for future cross-sectional analysis.

Composition: three proximal and three distal samples of kangaroo Achilles tendon and three AM and three PL samples of human ACL were immunostained for Type I and III collagen and histo-chemically stained (Curtis modified Verhoff van Gieson) for elastin. For immunostaining, sections were de-waxed and rehydrated, treated with Proteinase K (Dako # S3020, 1/10 for 30 mins at room temperature) and then Bovine Testicular Hyaluronidase (Sigma # H3505-5G 1000 units/ml in pH 5.0 phoshate buffer 0.1M for 2 hours at 37° C.) to expose antigens. The sections were then incubated overnight at 4° C. with primary antibodies (Abcam ab90395 mouse monoclonal anti-type-I collagen at 10 g/ml; Abcam ab7778 Rabbit polyclonal anti-type-Ill at 3.3 ug/ml; versus equivalent concentrations of Dako # X0931 mouse IgG or Dako X0936 Rabbit IgG, respectively, as negative controls), washed, localised using Dako EnVision and Immpact NovaRed according to the manufacturers' instructions and counterstained with Mayers haematoxylin.

Statistical analyses: differences in cell counts between regions within kangaroo Achilles tendon (proximal and distal) or human ACL (AM and PL) were compared using a paired or unpaired Students t-test, respectively. The same test was used to determine differences in cell counts between kangaroo Achilles tendon and human ACL (pooled regions in each tissue).

### Results & Discussion

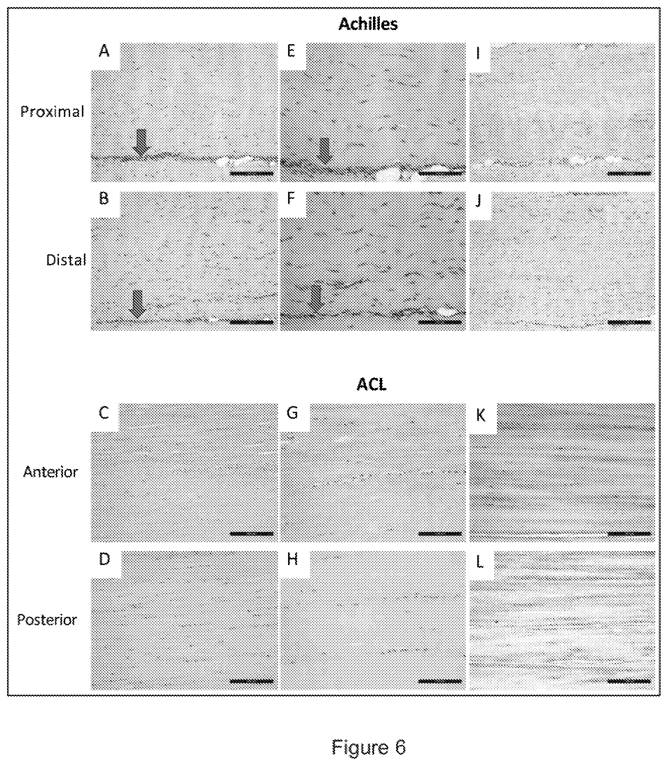

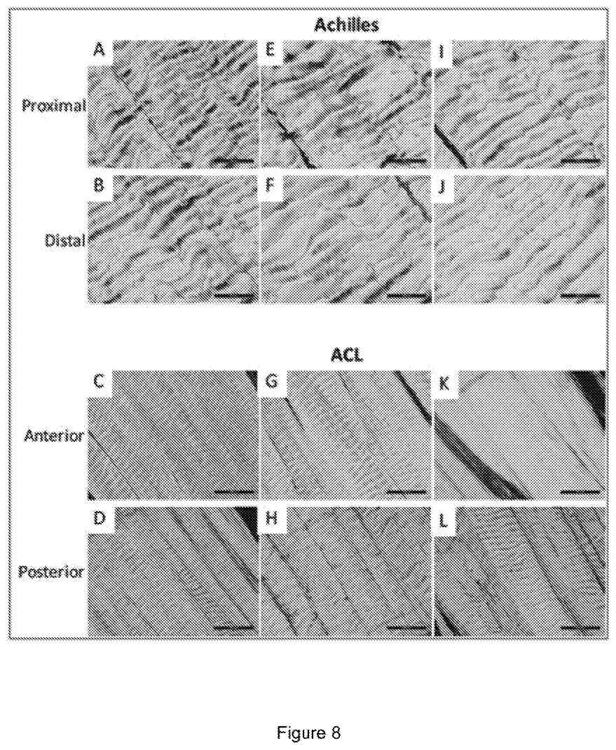

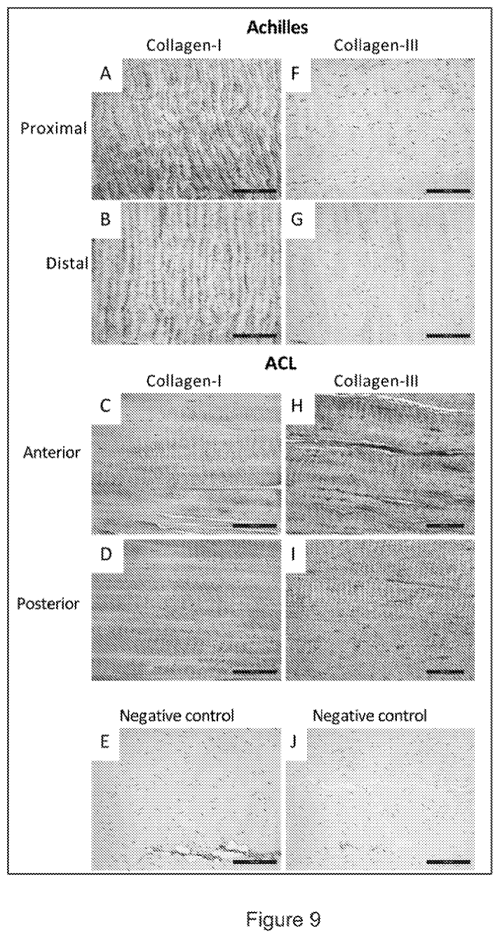

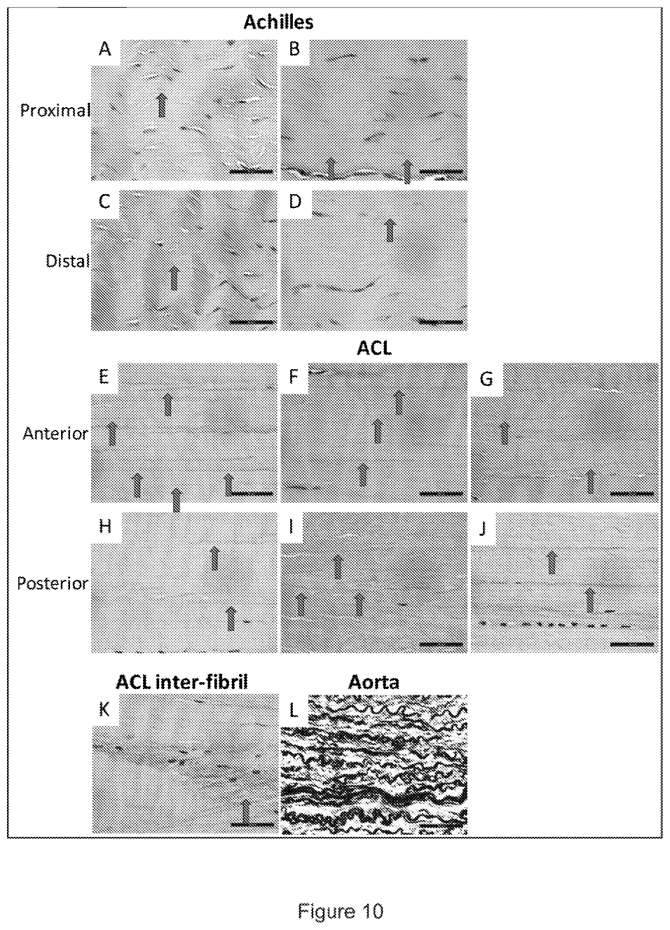

Histo-morphology and histo-chemical/immuno-histological staining can show variability between individual samples of a given tissue and also regionally within any single sample/section. FIGS. 6 and 9 are representative images showing the typical histology of a given tissue where it reasonable consistency was observed. FIGS. 8 and 10 are representative images showing the range of histology that was seen in that tissue.

The kangaroo Achilles tendon and human ACL were composed principally of dense longitudinally oriented collagen fibres, with no discernible difference between regions (proximal/distal or AM/PL) within either tissue (FIG. 6 A-H). There were, however, differences between the kangaroo Achilles tendon and human ACL. The kangaroo Achilles tendon had more cells, which were relatively evenly distributed through the tissue largely as single cells with an elongated nucleus and fibroblastic appearance (FIG. 6 A, B, E, F). The human ACL had fewer cells, which were often found in longitudinal clusters/columns, with many of the cells having a more rounded fibro-chondrocyte appearance (FIG. 6 C, D, G, H). The kangaroo Achilles tendon also had prominent interfibrillar zones with mesenchymal cell accumulation and occasional blood vessels that are typical of tendons (FIG. 6. A, B. E, F; indicated by arrows), which were largely absent or much smaller with fewer cells in the human ACLs.

In both the kangaroo Achilles tendon and the human ACL there were regions within individual samples with increased matrix proteoglycan, evidenced by metachromatic staining with Toluidine Blue (FIG. 6 I-L). These proteoglycan-rich areas were found in both regions of the kangaroo Achilles tendon and both the AM and PL bundles in the human ACL. However, staining tended to be more intense and longitudinally oriented in the human ACL compared with the more diffuse staining in the kangaroo Achilles tendon.

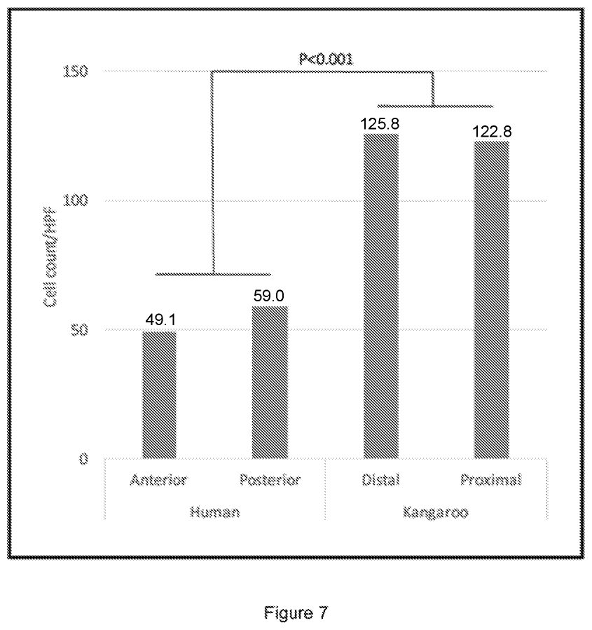

The apparently greater cellularity of the kangaroo Achilles tendon compared with the human ACL was borne out by cell counting. No difference was seen between regions in either tissue, but approximately twice as many cells/highpower field was observed in the kangaroo Achilles tendon compared with the human ACL (P<0.001; FIG. 7).

Polarized light microscopy of Picrosirius-red stained sections suggested differences in collagen fibre morphology and alignment between kangaroo Achilles tendon and human ACL and also between regions within both tissues (FIG. 8). The observed crimp pattern was consistently larger in the kangaroo Achilles tendon compared with the human ACL, although there seemed to be no difference between regions within either tissue. Collagen fibre alignment was variable within any given specimen and between individual samples of any one tissue (FIG. 8). However, there was generally better alignment in proximal compared with distal kangaroo Achilles tendon and in anterior compared with posterior human ACL. Finally, the best fibre alignment was seen in human ACL rather than kangaroo Achilles tendon (compare panels A and C in FIG. 8), although whether this was due to partial masking as a result of the larger crimp pattern in the latter is unclear.

Strong Collagen-I staining was observed throughout the kangaroo Achilles tendon and human ACL, with little or no consistent difference between tissues or between regions within either tissue (FIG. 9 A-D). In contrast, Collagen-III staining was much stronger in human ACL than in kangaroo Achilles tendon, although again there was no discernible difference between regions in either tissue (FIG. 9 F-I).

There was a marked difference in both the abundance and architecture of elastin micro-fibres between the kangaroo Achilles tendon and human ACL, although no difference was observed between regions within each tissue (FIG. 10). In the kangaroo Achilles tendon, the few elastin fibres identified were short and all were running parallel to the collagen (FIG. 10 A-D, indicated by arrows). In contrast, elastin fibres were readily identified in human ACL samples and were often considerably longer that those in kangaroo Achilles tendon samples (FIG. 10 E-J, indicated by arrows). While elastin fibres largely run parallel to collagen in human ACL, inter-fibrillar elastin fibres were also identified in this tissue running at an angle to the collagen (FIG. 10 K). The elastin micro-fibres in both kangaroo Achilles tendon and human ACL were of similar diameter, being considerably smaller than those in elastic tissues such as aorta (FIG. 10 L).

The overall morphological appearance of kangaroo Achilles tendon is consistent with other load-bearing and positional tendons. The higher number of cells in kangaroo Achilles tendon compared with human ACL may be due to the difference between tendon and ligament. It could also be associated with the expected reduction in cell number in older individuals (and thus a lower number in the human samples). The number of cells in the kangaroo Achilles tendon could have an impact on the conditions required for de-cellularization if necessary for its use as a graft in humans.

The above analysis does not take into account the inter-fibrillar tissue and mesenchymal and vascular cells therein, seen much more abundantly in kangaroo Achilles tendon compared with human ACL. This tissue and these cells do not represent an additional challenge in using kangaroo Achilles tendon as they are very typical of tendon tissue. Indeed, these structures in tendon may be advantageous for access of both the initial de-cellularizing agents and then the host blood vessels and re-populating fibroblasts.

Both kangaroo Achilles and human ACL are rich in type I collagen, which is the principal fibrillar collagen in tensile connective tissues. Differences in staining for type III collagen between Achilles and ACL could be associated with differential affinity/recognition by the antibody for kangaroo versus human type Ill collagen, although these proteins are generally well-conserved across species.

Overall the observed data indicate that the kangaroo Achilles tendon shows morphology, collagen architecture, collagen arrangement and composition similar to tendons in humans. The observed differences between kangaroo Achilles tendon and human ACL may therefore be associated differences between tendon and ligament tissue. Tendon tissue used as a graft for repairing ACL tissue typically undergoes a ligamentization process, which involves remodelling and change in collagen fibre types and development of both elastin and oxytalin fibres (Zaffagnini, S., De Pasquale, V., Marchesini Reggiani, L., Russo, A., Agati, P., Bacchelli, B., Marcacci, M., “Neoligamentization process of BTPB used for ACL graft: histological evaluation from 6 months to 10 years”, Knee, 2007, 14(2):87-93).

The xenografts and methods described herein are presented by way of example only and are not limiting as to the scope of the invention. Unless otherwise specifically stated, individual aspects and components of the xenografts and methods may be modified, or may have been substituted therefore known equivalents, or as yet unknown substitutes such as may be developed in the future or such as may be found to be acceptable substitutes in the future. The xenografts and methods may also be modified for a variety of applications while remaining within the scope and spirit of the claimed invention, since the range of potential applications is great, and since it is intended that the present xenografts and methods be adaptable to many such variations.

1. A xenograft for treating a tendon or ligament condition in a subject, wherein said xenograft comprises at least a portion of a macropod tendon.

2. The xenograft of claim 1, wherein the macropod tendon is an Achilles tendon.

3. The xenograft of claim 1, wherein the macropod tendon is a tail tendon.

4. The xenograft of claim 1, wherein the macropod is selected from the group consisting of red kangaroo (Macropus rufus), eastern grey kangaroo (Macropus giganteus), western grey kangaroo (Macropus fuliginosus), black wallaroo (Macropus bernardus), antilopine wallaroo (Macropus antilopinus), common wallaroo (Macropus robustus), agile wallaby (Macropus agilis), black-striped wallaby (Macropus dorsalis), red-necked wallaby (Macropus rufogriseus), swamp wallaby (Wallabia bicolor) and whiptail wallaby (Macropus parryi).

5. The xenograft of claim 1, wherein the macropod is selected from the group consisting of red kangaroo (Macropus rufus), eastern grey kangaroo (Macropus giganteus) and western grey kangaroo (Macropus fuliginosus).

6. The xenograft of claim 1, wherein the macropod is an eastern grey kangaroo (Macropus giganteus).

7. The xenograft of claim 1, which is non-immunogenic.

8. The xenograft of claim 1, wherein the subject is a mammal.

9. The xenograft of claim 8, wherein said mammal is a human.

10. The xenograft of claim 1, wherein said xenograft has been subjected to a decellularization process.

11. The xenograft of claim 1, wherein said portion of macropod tendon has an ultimate tensile strength of at least about 10 MPa.

12. The xenograft of claim 1, wherein said portion of macropod tendon has an ultimate tensile strength of at least about 20 MPa.

13. The xenograft of claim 1, wherein said portion of macropod tendon has an ultimate tensile strength of at least about 30 MPa.

14. The xenograft of claim 1, wherein said portion of macropod tendon has an elastic modulus of at least about 50 MPa.

15. The xenograft of claim 1, wherein said portion of macropod tendon has an elastic modulus of at least about 100 MPa.

16. The xenograft of claim 1, wherein said portion of macropod tendon has an elastic modulus of at least about 200 MPa.

17. A method of treating a tendon or ligament condition in a mammal, the method comprising implanting the xenograft of claim 1 at the site of said tendon or ligament in the mammal.

18. The method of claim 17, wherein said tendon or ligament condition is a tendon injury or ligament injury.

19. The method of claim 17, wherein said tendon or ligament is selected from the group consisting of anterior cruciate ligament (ACL), posterior cruciate ligament (PCL), lateral collateral ligament (LCL), medial collateral ligament (MCL), patellar ligament, palmar radiocarpal ligament, dorsal radiocarpal ligament, ulnar collateral ligament, radial collateral ligament, Achilles tendon, flexor tendon of the hand, flexor tendon of the foot, extensor tendon of the hand, extensor tendon of the foot, rotator cuff tendons of the shoulder, hip abductor tendons, deltoid ligament complex of the ankle, lateral ligament complex of the ankle, calcaneofibular ligament, patella tendon, quadriceps tendon, medial patellofemoral ligament of the knee and lateral patellofemoral ligament of the knee.

20. The method of claim 17, wherein the strength of the at least a portion of the macropod tendon is equal to or greater than the strength of a healthy native tendon or ligament and/or wherein the elastic modulus of the at least a portion of the macropod tendon is equal to or greater than the elastic modulus of a healthy native tendon or ligament.

21.-22. (canceled)
